(19) United States
(12) Patent Application Publication
(10) Pub. No.: US 2025/0128020 A1
(43) Pub. Date: Apr. 24, 2025

(54) STRUCTURAL GUIDEWIRE

(71) Applicant: Mark Wilson Ian Webster, Epsom (NZ)

(72) Inventor: Mark Wilson Ian Webster, Epsom (NZ)

(21) Appl. No.: 18/924,472

(22) Filed: Oct. 23, 2024

(30) Foreign Application Priority Data
Oct. 24, 2023 (NZ) 804810A

(51) Int. Cl. A61M 25/00; A61M 25/09

(52) U.S. Cl. CPC A61M 25/00; A61M 2025/09075

(57) ABSTRACT

A guidewire for intravascular procedures is provided. The guidewire may include a guidewire shaft and a distal end. The guidewire shaft may include a proximal shaft portion that is substantially straight and an intermediate portion integral with or connected to the proximal shaft portion. The intermediate portion may have a meandering shape in at least one first plane. The distal end may be connected to the intermediate portion of the guidewire shaft and have a curved shape in a second plane different from the at least one first plane of the meandering shape of the intermediate portion.

### CROSS-REFERENCE TO RELATED APPLICATION

This application claims priority from N.Z. Provisional Patent Application No. 804810A, filed Oct. 24, 2023, and entitled “Structural guidewire,” which is herein incorporated by reference in its entireties.

### FIELD OF THE INVENTION

The invention relates to guidewires for intravascular procedures, and more specifically, to structural guidewires used in transcatheter aortic valve replacement (TAVR) procedures that include a meandering-shaped intermediate region to facilitate valve crossing and prosthetic valve alignment.

### BACKGROUND

Guidewires are essential tools in various intravascular procedures, playing an important role in navigating through blood vessels and facilitating the delivery of medical devices. These thin, flexible wires have been used for decades in interventional cardiology, radiology, and other minimally invasive procedures. Traditionally, guidewires have been designed with a straight shaft and a curved or J-shaped tip to aid in navigation. However, as medical procedures have become more complex, particularly in the field of structural heart interventions, there has been a growing need for more specialized guidewire designs.

One area where guidewires play a particularly critical role is in transcatheter aortic valve replacement (TAVR) procedures. TAVR has emerged as a less invasive alternative to open-heart surgery for treating severe aortic stenosis, especially in high-risk patients. The success of TAVR procedures heavily relies on the ability to accurately position and deploy the prosthetic valve, which in turn depends on the performance of the guidewire used. Thus, there are significant challenges in navigating heavily calcified and stenosed aortic valves, as well as in achieving optimal alignment of the prosthetic valve with the native annulus.

### BRIEF SUMMARY

The present disclosure seeks to provide a guidewire for intravascular procedures with improved navigation and positioning capabilities, and methods for its use and manufacture. The guidewire features a unique meandering shape in an intermediate portion to facilitate centering in blood vessels, navigating through heavily calcified and stenosed valves, and optimal alignment and deployment of prosthetic medical devices. The meandering shape is designed to operate in at least one plane different from that of the distal end, allowing for enhanced maneuverability and control during complex procedures such as transcatheter aortic valve replacement (TAVR).

In one aspect, a guidewire for intravascular procedures is provided. The guidewire includes a guidewire shaft including a proximal shaft portion being substantially straight and an intermediate portion integral with or connected to the proximal shaft portion, the intermediate portion having a meandering shape in at least one first plane. The guidewire further includes a distal end connected to the intermediate portion of the guidewire shaft, the distal end having a curved shape in a second plane different from the at least one first plane of the meandering shape of the intermediate portion.

In another aspect, a method of advancing a guidewire through a blood vessel is provided. The method includes inserting a guidewire into a blood vessel of a human body, wherein the guidewire includes a meandering shaped region. The method further includes advancing the guidewire through the blood vessel toward a target treatment tissue, and facilitating centering of the guidewire in the blood vessel by engaging the meandering shape against a wall of the blood vessel or a wall of the target treatment tissue.

In a further aspect, a method of manufacturing a guidewire for intravascular procedures is provided. The method includes forming a guidewire shaft including a proximal shaft portion and an intermediate portion, the forming of the guidewire shaft including shaping the proximal shaft portion to be substantially straight and shaping the intermediate portion of the guidewire shaft to have a meandering shape in at least one first plane. The method further includes forming a distal end, including shaping the distal end to have a curved shape in a second plane different from the at least one first plane of the meandering shape of the intermediate portion.

In some examples, the meandering shape is operable to centralize the guidewire in a valve having commissures and move at least a portion of the guidewire out of the commissures.

In some examples, the curved shape of the distal end includes a pigtail-shape or an S-shape.

In some examples, the distal end includes a core made of stainless steel or of nitinol.

In some examples, the meandering shape of the intermediate portion includes at least one of: a C-shape, or an S-shape.

In some examples, the meandering shape of the intermediate portion includes a three-dimensional spiral shape.

In some examples, the meandering shape of the intermediate portion is offset from a longitudinal axis of the guidewire.

In some examples, the meandering shape of the intermediate portion is collinear or coincident with the longitudinal axis of the guidewire.

In some examples, the intermediate portion has a length of 150-300 mm.

In some examples, the guidewire shaft has a stiffness of 1.3-3.6 N/mm.

In some examples, the guidewire has an overall length of 2500-3000 mm.

In some examples, the guidewire has a diameter of 0.027-0.035 inches.

In some examples, the second plane is perpendicular to one of the at least one first plane.

In some examples, the guidewire further includes a transition zone disposed between the proximal shaft portion and the distal end, the transition zone including: a tapered remote section of the intermediate portion with a reduced diameter; a tapered proximal section of the distal end with a reduced diameter; and an outer sleeve enclosing and connecting the tapered remote section of the intermediate portion and the tapered proximal section of the distal end.

In some examples, the tapered remote section of the intermediate portion and the tapered proximal section of the distal end each have a D-shaped cross-section, with flat surfaces of the D-shaped cross-sections facing each other and curved surfaces of the D-shaped cross-sections contacting the outer sleeve.

In some examples, the blood vessel is an aorta and the target treatment tissue is an aortic valve, the method further including: advancing a transcatheter aortic valve replacement (TAVR) delivery system over the guidewire and deploying a prosthetic aortic valve into the aortic valve.

In some examples, the meandering shape has an S-shaped curve disposed along an intermediate portion of the guidewire, the intermediate portion disposed between a proximal shaft portion and a distal end of the guidewire.

In some examples, the method further includes: positioning a distal end of the guidewire in a heart chamber and positioning the meandering shape across the target treatment tissue, wherein the target treatment tissue is an aortic valve, and the heart chamber is a left ventricle.

In some examples, the target treatment tissue is a valve, the method further including: advancing or withdrawing the guidewire to adjust a position of the meandering shape relative to the valve to change an angle of approach for a valve delivery system, the angle of approach corresponding to the angle at which the valve delivery system approaches the valve during deployment; and advancing the valve delivery system at the changed angle of approach over the guidewire and across the valve.

In some examples, the method further includes: advancing, withdrawing, or rotating the guidewire to align a distal ring marker of the valve delivery system with an annular plane of the valve.

Other technical features may be readily apparent to one skilled in the art from the following figures, descriptions, and claims.

### BRIEF DESCRIPTION OF THE SEVERAL VIEWS OF THE DRAWINGS

Throughout the drawings, reference numbers may be re-used to indicate correspondence between referenced elements. The drawings are provided to illustrate examples of the subject matter described herein and not to limit the scope thereof.

FIG. 1 illustrates a side view of a structural guidewire, in accordance with some examples.

FIG. 2 illustrates a perspective view of a structural guidewire, in accordance with some examples.

FIGS. 3A-3B illustrate side and front views, respectively, of a structural guidewire, in accordance with some examples.

FIG. 4 illustrates a side view of a structural guidewire, in accordance with some examples.

FIG. 5 illustrates a structural guidewire including a transition zone, in accordance with some examples.

FIGS. 6A-6C illustrate side views and a cross-sectional view of a transition zone between a guidewire shaft and a distal end of a structural guidewire, in accordance with some examples.

FIG. 7 illustrates a schematic diagram of a heart including valves, vessels, and chambers, in accordance with some examples.

FIG. 8 illustrates visual alignment of a plane of a medical device delivery system with that of a valve, in accordance with some examples.

FIG. 9 illustrates a flowchart of a method of using a structural guidewire, in accordance with some examples.

FIG. 10 illustrates a flowchart of a method of manufacturing a structural guidewire, in accordance with some examples.

FIG. 11 illustrates medical images indicating heavy calcification around aortic valves, in accordance with some examples.

### DETAILED DESCRIPTION

The present disclosure provides a structural guidewire (also referred to simply as a “guidewire” herein) for intravascular procedures with improved navigation and positioning capabilities. The structural guidewire may be particularly useful for transcatheter aortic valve replacement (TAVR) procedures but can also be used for other procedures that involve navigating through complex vascular structures and precise positioning of medical devices, such as coronary interventions, peripheral vascular procedures, neurovascular treatments, etc.

In some examples, a structural guidewire includes a guidewire shaft having a proximal shaft portion, an intermediate portion, and a distal end. The proximal shaft portion is substantially straight, providing stability and trackability during insertion and advancement through blood vessels. In some examples, the intermediate portion, integral with or connected to the proximal shaft portion, features a unique meandering shape in at least one plane. The meandering shape may take the form of a C-shape, S-shape, a three-dimensional spiral shape, or the like, or any combination thereof. The meandering shape may facilitate centering of the structural guidewire in blood vessels and navigate through heavily calcified and stenosed valves. Additionally, the meandering shape allows for improved support and control during the advancement of delivery systems.

In some examples, the distal end of the guidewire is either integral with or connected to the intermediate portion and has a curved shape, such as a pigtail-shape or an S-shape. The curved shape of the distal end may be in a plane different from the at least one plane of the meandering shape of the intermediate portion. This configuration may also allow for enhanced maneuverability and control during complex procedures. The curved distal end may provide a stable anchor within heart chambers, reducing the risk of cardiac perforation. The guidewire shaft may be made of stainless steel (or have a stainless steel core) to provide strength and pushability and the curved distal end may be made of a different material than the shaft, such as nitinol, or have a nitinol core, to provide flexibility, trackability, and shape retention properties. However, they can be made of other types of same or different materials and shall not be limiting here.

In some examples, a structural guidewire may have specific dimensions and properties tailored for satisfactory performance. For example, the intermediate portion may have a length of 150-300 mm, the guidewire shaft may have a stiffness of 1.3-3.6 N/mm, and the overall length of the guidewire may be 2500-3000 mm with a diameter of 0.027-0.035 inches. It should be noted that other dimensions or properties are also within the protection scope of this disclosure, as the specific requirements may vary depending on the particular intravascular procedure and patient anatomy.

In some examples, if the distal end of the guidewire is not integral with the guidewire shaft, the guidewire shaft and the distal end may be connected at a transition zone. The transition zone may include a tapered remote section of the guidewire shaft with a reduced diameter, a tapered proximal section of the distal end with a reduced diameter, and an outer sleeve enclosing these tapered sections to connect the guidewire shaft with the distal end.

The guidewire can be used in various intravascular procedures. An example method of using the guidewire includes inserting the guidewire into a blood vessel and advancing it toward a target treatment tissue. The method may further include advancing, withdrawing, or rotating the guidewire to adjust the position of the meandering shape relative to the target treatment tissue, facilitating optimal alignment and approach angles. For TAVR procedures, the method may further include advancing a TAVR delivery system over the guidewire and deploying a prosthetic aortic valve.

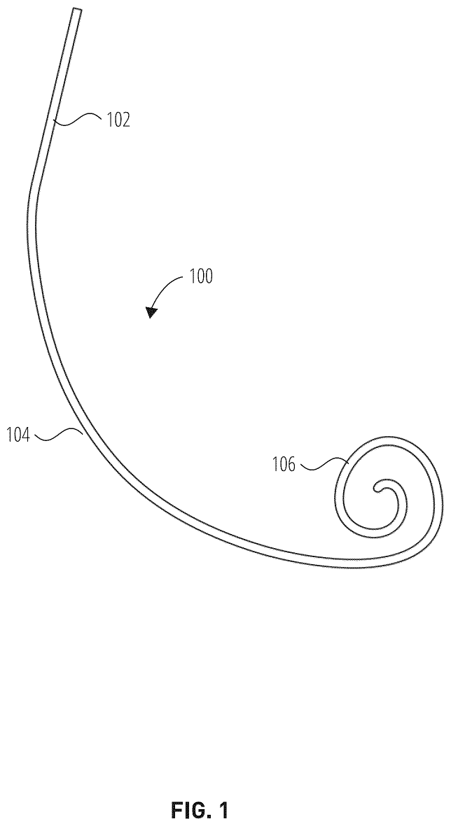

FIG. 1 illustrates a side view of a structural guidewire 100, in accordance with some examples. The guidewire 100 may include three main sections: a proximal shaft portion 102, an intermediate portion 104, and a distal end 106. The proximal shaft portion 102 and the intermediate portion 104 may collectively be referred to as a guidewire shaft in the present disclosure.

In some examples, the proximal shaft portion 102 may be substantially straight, providing stability, pushability, and trackability during insertion and advancement through blood vessels. The straight proximal shaft portion 102 may allow for transmission of force from a proximal end (e.g., operator) to the intermediate portion 104 (and then the distal end 106) of the guidewire 100, facilitating navigation through complex vascular structures.

In some examples, the intermediate portion 104 may feature a unique shape that deviates from the straight proximal portion. In FIG. 1, the intermediate portion 104 is shown as a gentle curve that transitions from the straight proximal portion 102 to a more significantly curved distal end 106. Other shapes or properties of the intermediate portion 104 are possible, for example as described elsewhere in the present disclosure. The proximal shaft portion 102 and the intermediate portion 104 may be designed for a balance between the pushability provided by the straight section in the proximal shaft portion 102 and the navigational advantages (trackability) offered by the curved sections in the intermediate portion 104.

In some examples, the distal end 106 of the guidewire 100 may have a curved shape, such as a pigtail shape as illustrated in FIG. 1. The curved shape of the distal end 106 may be designed to be in a different plane from the plane of the intermediate portion 104. The curved shape of the distal end 106 may provide a stable anchor within heart chambers, reducing the risk of cardiac perforation. The curved shape of the distal end 106 may also enhance maneuverability and control during complex procedures and offer a soft, atraumatic tip for navigating delicate vascular structures. For example, the curved shape of the distal end 106 may help the guidewire smoothly pass through the aortic arch between descending aorta and ascending aorta.

In some examples, the materials used for different sections of the guidewire 100 may be carefully selected to optimize performance and functionality for intravascular procedures. The below configuration is merely an example and shall not be limiting.

In some examples, the guidewire shaft, including the proximal shaft portion 102 and the intermediate portion 104, may be constructed primarily of high-tensile stainless steel, such as 304V or 316LVM, to provide the necessary strength, pushability, and torque transmission required for navigating complex vascular anatomy. The stainless steel core may be designed with varying degrees of stiffness along its length, potentially achieved through tapering or selective heat treatments, to provide a stiffness gradient that balances pushability in the proximal section with increased flexibility towards the distal end. The distal end 106 may be made from stainless steel or from nitinol, a nickel-titanium alloy with good elasticity and shape memory characteristics. This material choice allows the distal end 106 to maintain its pre-set curved shape (e.g., pigtail or S-shape) while still being able to straighten when navigating tortuous vessels. A nitinol component may undergo specific heat treatments to fine-tune its mechanical properties and shape-setting behavior. Additionally, the transition between the stainless steel shaft and the nitinol distal end may incorporate a carefully designed joint, such as a sleeve or welded connection, to ensure a smooth transition in flexibility and maintain structural integrity. To enhance the guidewire's overall performance, various surface treatments and coatings may be applied. The entire length of the guidewire may be coated with a hydrophilic polymer to reduce friction and improve lubricity, facilitating smoother navigation through blood vessels. Furthermore, the guidewire 100, or a part thereof, may include composite structures, such as a nitinol core with a stainless steel outer coil or a stainless steel core with a polymer outer coil in certain sections, to combine the benefits of different materials and optimize the balance between flexibility, pushability, and kink resistance.

In some examples, the guidewire 100 may have an overall length ranging from 2500 mm to 3000 mm but this is not limiting. Any length that allows for sufficient reach during complex intravascular procedures while providing reasonable external length for manipulation by the operator can be chosen. The diameter of the guidewire 100 may fall within the range of 0.027-0.035 inches, but this is not limiting. Any diameter that may provide a balance between flexibility and support for various intravascular applications may be chosen.

In some examples, the intermediate portion 104 of the guidewire 100, which features the unique shape, may have a length ranging from 150 mm to 300 mm but this is not limiting. More specific ranges within this spectrum could include 150-200 mm, 200-250 mm, or 250-300 mm, depending on the specific design requirements and intended use. Any length of the intermediate portion 104 that can provide adequate coverage of a target treatment tissue during medical procedures can be chosen. The distal end 106 with the curved shape (e.g., pigtail or S-shape) may have a length of approximately 150 mm, but this is not limiting too.

In some examples, the guidewire shaft (including the proximal shaft portion 102 and the intermediate portion 104) may have a stiffness ranging from 1.3 N/mm to 3.6 N/mm, but this is not limiting. This range can be further subdivided into categories such as: medium stiffness: 1.3-1.8 N/mm, medium-high stiffness: 1.8-2.5 N/mm, high stiffness: 2.5-3.2 N/mm, extra high stiffness: 3.2-3.6 N/mm. The specific stiffness within these ranges may be tailored to provide optimal performance for different intravascular procedures, with medium (or low) stiffness offering more flexibility for navigation and higher stiffness providing better support for device delivery.

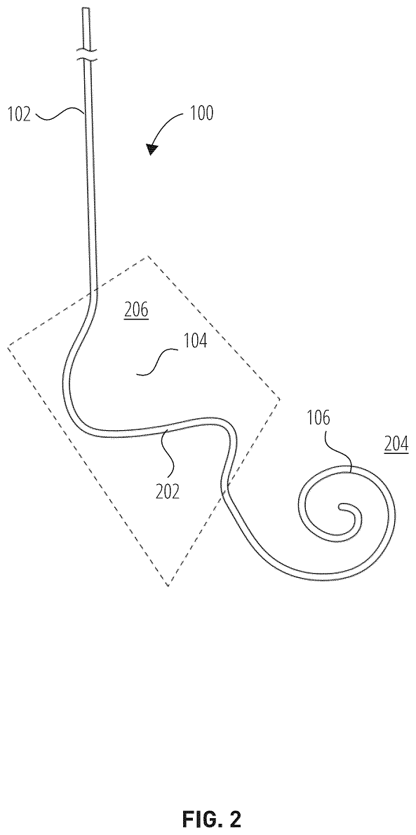

FIG. 2 illustrates a perspective view of a structural guidewire 100 in accordance with some examples. Similar to FIG. 1, the guidewire 100 may include a proximal shaft portion 102, an intermediate portion 104, and a distal end 106. Instead of being a gentle curve as illustrated in FIG. 1, the intermediate portion 104 may include a meandering shape 202. The meandering shape 202 may take various forms, such as a C-shape, an S-shape, or a three-dimensional spiral shape, or the like, or any combination thereof. The meandering shape can provide enhanced navigational capabilities and facilitates centering within blood vessels through multiple points of contact with the vessel wall, providing better stability and positioning. The meandering shape 202 may lie in one plane, but in some examples, it may include multiple planes, creating a more complex three-dimensional structure.

As shown in FIG. 2, the plane 204 of the curved shape of the distal end 106 (e.g., pigtail or S-shape) may be different from the plane 206 of the meandering shape 202 of the intermediate portion 104. In some examples, the plane 204 of the distal end 106 may be perpendicular to the plane 206 of the meandering shape 202, or they may form any other angle therebetween that can provide optimal performance for specific procedures. This configuration of different planes 204 and 206 allows for improved three-dimensional navigation and positioning within complex vascular structures.

The guidewire 100 can be manipulated by advancing, withdrawing, or rotating to adjust the position of the meandering shape 202 relative to a target treatment tissue. This manipulation allows for precise control and optimization of the guidewire's position during procedures. For instance, in transcatheter aortic valve replacement (TAVR) procedures, advancing or withdrawing the guidewire 100 a short distance can change the intermediate portion 104 of the guidewire 100 and its angulation at the level of the aortic valve. This adjustment may facilitate crossing the aortic valve with a valvuloplasty balloon or the aortic valve delivery system. Additionally, this adjustment can help in navigating through heavily calcified valves or in optimizing the approach angle for valve deployment. Furthermore, advancing, withdrawing, or rotating the guidewire 100 can alter the plane of the distal end of the delivery system once it has crossed the aortic valve. This manipulation may allow for better alignment of the distal end of the valve frame with the annular plane, which is important for optimal prosthetic valve deployment.

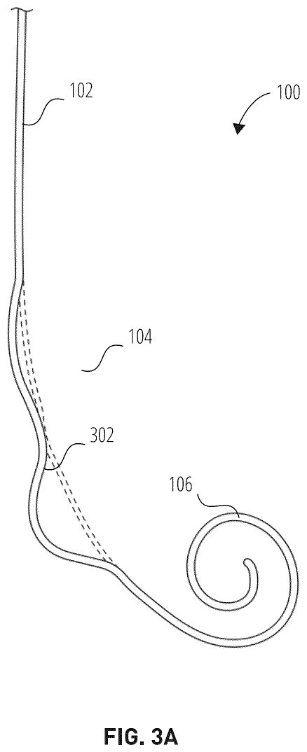

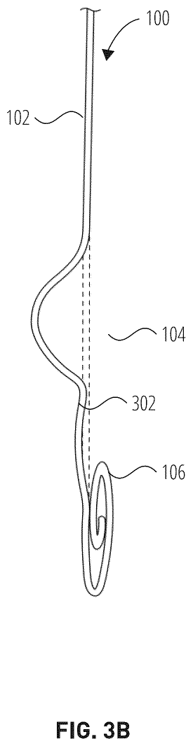

FIG. 3A and FIG. 3B illustrate side and front views, respectively, of a structural guidewire 100 in accordance with some examples. Similar to FIG.1 and FIG. 2, the guidewire 100 includes a proximal shaft portion 102, an intermediate portion 104, and a distal end 106. As shown in FIG.3A and FIG. 3B, the intermediate portion 104 may feature a complex meandering shape 302 that includes two C-shaped curves that are offset or non-symmetrical about the longitudinal axis of the guidewire 100. The C-shaped design of the meandering shape 302 may offers several benefits. For example, the C-shape may provide a more gradual curve, potentially allowing for smoother navigation through tortuous vessels or calcified valves. It could also possibly offer better centering capabilities within blood vessels by providing strategic points of contact against the vessel wall. Additionally, the C-shape may serve to distribute forces more evenly along the guidewire, potentially reducing the risk of kinking or damage during use.

The off-centered or non-symmetrical arrangement of the C-shaped curves may facilitate crossing of heavily calcified valves by providing a non-uniform approach angle. An off-centered design might improve alignment of the delivery system with the annular plane of the valve, which is important for optimal prosthetic valve deployment in TAVR procedures. Furthermore, the asymmetrical design may allow for multiple approach angles by rotating the guidewire, providing more options for navigating challenging anatomies.

While the meandering shape 302 offers potential benefits for being off-centered or non-symmetrical, it should be noted that the 3-dimensional spiral shape 402 can also be centered along the longitudinal axis of the guidewire. The centered configuration may be preferred in certain clinical scenarios or for specific anatomical considerations. The choice between offset and centered configurations may depend on the particular procedural requirements and patient anatomy and they are both within the protection scope of the present disclosure.

Similar to FIG. 2, the plane of this distal curve may be different from the planes of the C-shaped curves of the meandering shape 302, allowing for improved three-dimensional navigation and positioning within complex vascular structures.

In some examples, multiple guidewires may be used together. For instance, a guidewire system (not shown in FIGs) may include a main guidewire and an auxiliary guidewire. Each of the two guidewires may include a proximal shaft portion, an intermediate portion with a meandering shape, and a distal end. The two guidewires can be connected in various ways and shall not be limiting. One possible connection is that the meandering shape of the main guidewire interacts with the curved section of the distal end of the auxiliary guidewire. This interaction could be designed to allow for temporary coupling or sliding contact between the two guidewires, enabling coordinated movement while maintaining individual maneuverability. Alternatively, the guidewires could be permanently connected at a specific point, such as around the end of meandering shape of the main guidewire, using a coupling mechanism or a shared outer sleeve. In some examples, the meandering shape of the auxiliary guidewire may lie in a plane that is different from the plane of the meandering shape of the main guidewire. The distal end of the main guidewire may be in yet another plane, potentially different from one or both of the planes of the meandering shapes of the guidewires. This multi-planar configuration could enhance the system's ability to navigate complex three-dimensional vascular structures.

In some examples, the intermediate portions of the two guidewires could incorporate multiple C-shaped curves, S-shaped curves, or a combination thereof, or any other 3-dimensional meandering shapes to offer more complex navigation options to comply with the shapes of different vessels.

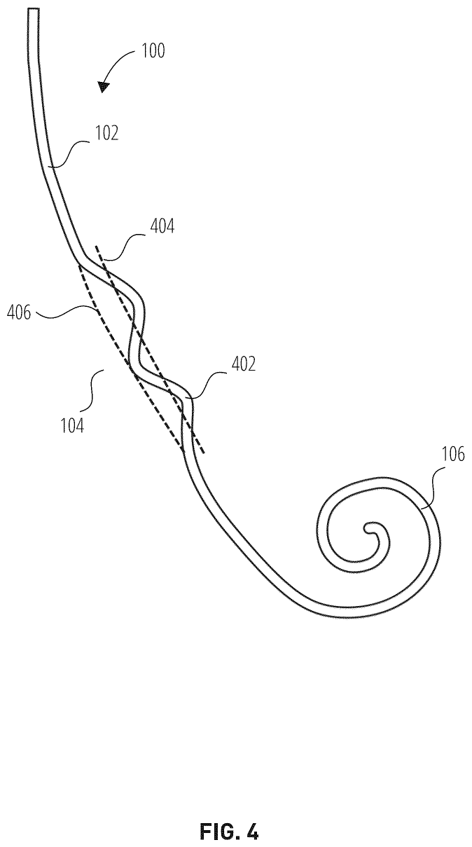

FIG. 4 illustrates a side view of a structural guidewire 100, in accordance with some examples. Similar to FIG. 1 to FIG. 3B, the guidewire 100 includes a proximal shaft portion 102, an intermediate portion 104, and a distal end 106. As shown in FIG. 4, the intermediate portion 104 may include a 3-dimensional spiral shape 402. The 3-dimensional spiral shape 402 may be offset from the other parts of the guidewire 100, e.g., the centerline 404 of the 3-dimensional spiral shape 402 does not collineate or coincide with the longitudinal axis 406 of the guidewire 100. In some examples, the term “spiral shape” refers to multiple continuous spiral-like curves that extend in three dimensions, creating a helical structure along the length of the intermediate portion 104. The term “3-dimensional,” unlike a 2-dimensional shape which is flat or lies in a same plane, occupies marginally a cylindrical space. Such a 3-dimensional spiral shape 402 may allow the guidewire 100 to navigate and conform to the complex, curved anatomy of blood vessels effectively. This 3-dimensional configuration enables the guidewire 100 to make multiple points of contact with the vessel walls, potentially improving its centering capabilities and stability within the blood vessel. The spiral shape can distribute forces more evenly along the guidewire 100′s length, which may reduce the risk of kinking or damage during use. Additionally, the 3-dimensional nature of the spiral allows for a more versatile range of motion and approach angles.

While the 3-dimensional spiral shape 402 is exemplified to be off-centered or offset from the longitudinal axis 406 of the guidewire 100, it should be noted that the 3-dimensional spiral shape 402 can also be centered along the longitudinal axis 406 of the guidewire 100. The choice between offset and centered configurations may depend on the particular procedural requirements and patient anatomy and they are both within the protection scope of the present disclosure.

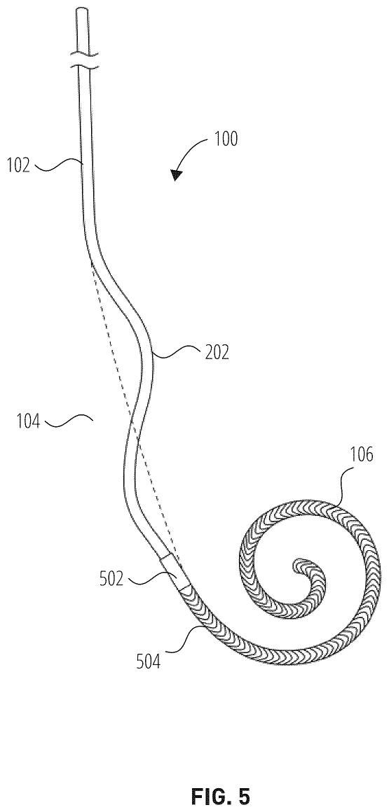

FIG. 5 illustrates a structural guidewire 100 including a transition zone 502, in accordance with some examples. The guidewire 100 retains the main features previously described, including a proximal shaft portion 102, an intermediate portion 104, and a distal end 106. As shown in FIG. 5, the guidewire 100 may further include a transition zone 502 between the intermediate portion 104 and the distal end 106. The transition zone 502 may incorporate a joint or coupling mechanism to ensure a smooth transition between different sections of the guidewire. In some examples, the guidewire 100 may use different materials for optimal performance. The proximal shaft portion 102 and intermediate portion 104 could be made of high-tensile stainless steel, while the distal end 106 may use nitinol for enhanced flexibility and shape memory.

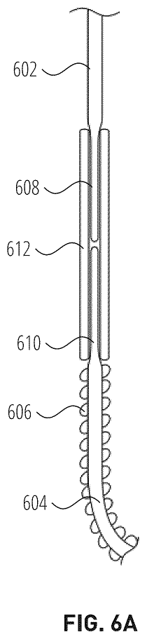

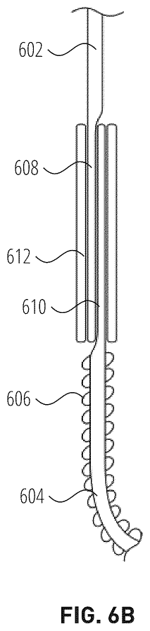

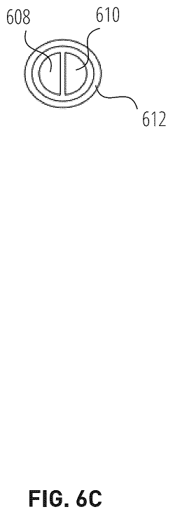

One potential design for the transition zone 502 could involve a gradual tapering of the intermediate portion 104 and the distal end 106, where the diameter of the wire decreases so that an outer sleeve can be used to enclose and connect them. Details of this design may be found in FIG. 6 and the descriptions thereof. Alternatively, the transition zone 502 is created by joining the intermediate portion 104 and the distal end using a joining technique such as friction welding, laser welding, etc.

In some examples, the transition zone 502 is integrally manufactured with the rest of the guidewire through advanced techniques such as laser cutting or 3D printing of metallic materials. These methods may allow for precise control over the geometry and material properties of the transition zone, potentially creating complex structures that are difficult to achieve with traditional manufacturing methods.

In some examples, a flat wire coil 504 is wound around the core wire in the transition zone 502 towards the distal end 106. In some examples, the guidewire 100 can include micro-engineered surface textures or patterns. These formations may be designed to interact with blood flow in specific ways, potentially reducing friction or enhancing the guidewire 100's ability to maintain its position within a vessel. Such surface modifications could be achieved through techniques like laser etching or chemical treatment.

FIGS. 6A-6C illustrate side views and a cross-sectional view of a transition zone (e.g., transition zone 502) between a guidewire shaft (e.g., intermediate portion 104) and a distal end (e.g., distal end 106) of an example guidewire 100, in accordance with some examples. The transition zone may include a remote section 602 of the intermediate portion 104, a proximal section 604 of the distal end 106, and an outer sleeve 612. The remote section 602 of the intermediate portion 104 may include a tapered section 608 with a reduced diameter. The proximal section 604 of the distal end 106 may include a tapered section 610, which also has a reduced diameter.

As shown in FIG. 6A, the two tips of the tapered sections 608 and 610 may contact each other and be enclosed and thereby firmly connected by an outer sleeve 612. As shown in FIGS. 6B and 6C, the tapered sections 608 and 610 may each have a D-shaped cross-section. The flat surfaces of these D-shaped cross-sections may face each other, while the curved surfaces contact the outer sleeve 612. The proximal section 604 of the distal end 106 of the guidewire 100 may include a wire coil 606 winding around the core of that section of the guidewire 100.

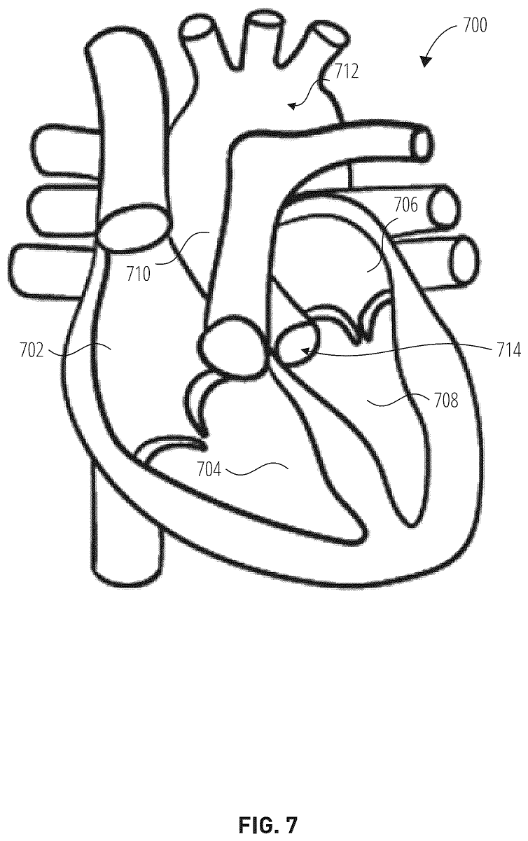

FIG. 7 illustrates a schematic diagram of a heart 700 including valves, vessels, and chambers, in accordance with some examples. As shown in FIG.7, the heart 700 includes a right atrium 702, a right ventricle 704, a left atrium 706, and a left ventricle 708. The heart 700 functions as a pump to circulate blood throughout the body. Deoxygenated blood enters the right atrium 702 and flows into the right ventricle 704, which pumps the blood to the lungs for oxygenation. Oxygenated blood returns to the left atrium 706 and enters the left ventricle 708. The left ventricle 708 then pumps the oxygenated blood through the aortic valve 714 into the aorta 710 for distribution to the rest of the body.

Transcatheter Aortic Valve Replacement (TAVR) is a minimally invasive procedure used to treat aortic valve stenosis. In TAVR, a catheter-based system is used to deliver and deploy a prosthetic valve within the diseased native aortic valve. The procedure typically involves advancing a guidewire through the aorta and across the stenotic aortic valve, followed by the delivery of a balloon-expandable or self-expanding prosthetic valve over the guidewire.

In an TAVR procedures, the guidewire travels and navigates through the aorta as follows:
1. The guidewire is typically inserted into a large artery, often the femoral artery in the groin.
2. The guidewire is then advanced through the iliac artery and into the abdominal aorta.
3. The guidewire continues up through the thoracic aorta and into the aortic arch 712.
4. From the aortic arch 712, it takes a turning and is maneuvered through the aortic valve 714 into the left ventricle 708.
5. Once in position, the guidewire provides a stable rail over which the TAVR delivery system can be advanced to deploy the prosthetic valve.

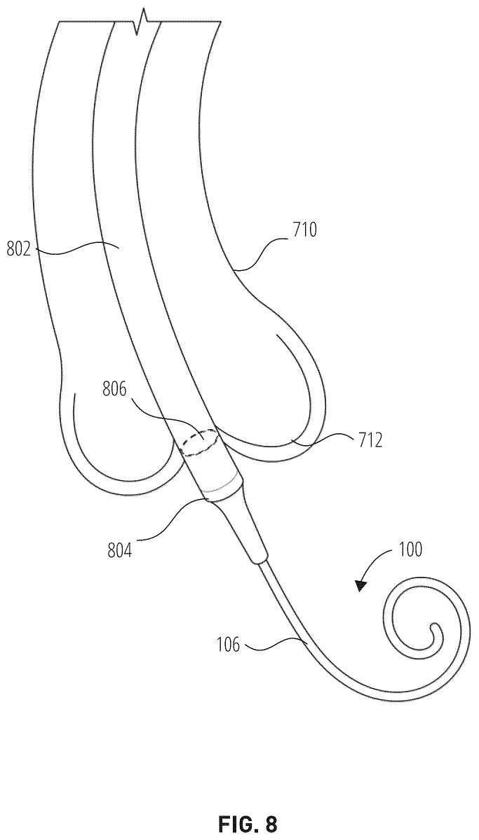

In some examples, a structural guidewire 100 described in the present disclosure can greatly smooth the TAVR procedure in several ways:
1. The meandering shape (e.g., meandering shapes 202, 302, 402) of the intermediate portion 104 helps to center the guidewire within the aorta and facilitates crossing the stenotic aortic valve. This centering effect can move the wire out of the commissures between valve leaflets, potentially reducing the risk of getting caught on calcified areas.
2. By advancing or withdrawing the guidewire a short distance, different portions of the meandering shape can be positioned at the level of the aortic valve.
3. The multi-planar configuration of the guidewire, with the meandering shape in at least one plane and the distal end 106 in another plane, enhances the ability to navigate the complex three-dimensional structure of the aortic arch and valve.
4. Once the delivery system has crossed the valve, advancing, withdrawing, or rotating the guidewire can help align the distal end of the delivery system with the annular plane of the valve. This can improve the positioning and deployment of the prosthetic valve. Further details on this aspect may be found in FIG. 8 and the descriptions thereof.
5. The transition zone between the intermediate portion 104 and distal end 106 provides a smooth flexibility transition, which can enhance force transmission while maintaining necessary flexibility for navigating tortuous vessels.
6. The pigtail or S-shaped distal end 106 provides a stable anchor within the left ventricle 708, reducing the risk of cardiac perforation during the procedure.
7. As the guidewire is advanced from the thoracic aorta into the aortic arch, the curved shape of the distal end 106 can reduce the risk of the guidewire scraping against the vessel wall or getting caught on anatomical structures within the arch.

Similar or different procedures on the same or different organs may be smoothed and benefited by the guidewire 100 and the use of the guidewire 100 is not limited to TAVR procedures. Some example procedures may include but are not limited to mitral valve repair or replacement, tricuspid valve repair or replacement, pulmonary valve repair or replacement, coronary artery interventions, peripheral vascular interventions, and neurovascular interventions.

FIG. 8 illustrates visual alignment of a plane of a medical device delivery system with that of a valve, in accordance with some examples. As shown in FIG. 8, a guidewire 100 may be deployed in a configuration required for TAVR procedures. Specifically, the meandering shape of the intermediate portion 104 of the guidewire 100 is positioned across the aortic valve 714 and the distal end 106 of the guidewire 100 is positioned in the left ventricle 708. The TAVR delivery system (such as the catheter 802) may take the guidewire 100 as a rail through aorta 710 towards the aortic valve 714. Before a prosthetic valve is deployed, it is important to verify that the distal marker 804 of the TAVR delivery system is aligned with the annulus plane 806 of the aortic valve 714. Benefited from the meandering shape and the multi-planar configuration of the guidewire 100, the guidewire 100 can be advanced, withdrawn, or rotated to alter the plane of the distal end of the delivery system so that a plane of circular marker 804 positioned in association with the structural guidewire 100 can be aligned with the annulus plane 806 of the aortic valve 714. In some examples, the circular marker 804 appears as a full ring when seen orthogonally, or a straight line when seen side-on, or an oval of differing sizes when seen at intermediate positions between the orthogonal and side-on views. A position of the structural guidewire 100, or a portion of it, may be adjusted or reoriented accordingly based on a visual alignment of the marker 804.

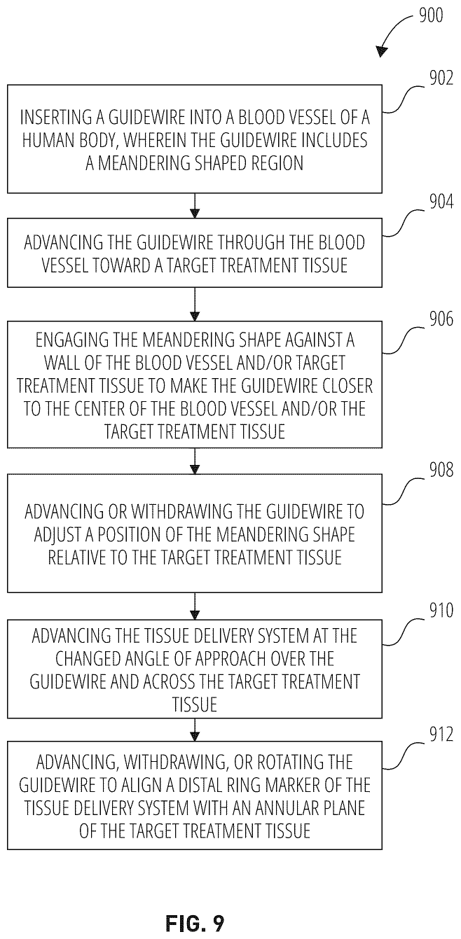

FIG. 9 illustrates a flowchart of a method 900 of using or deploying a structural guidewire 100, in accordance with some examples.

At operation 902, a user (e.g., an operator, a doctor) may insert the guidewire 100 into a blood vessel of a human body. The guidewire 100 may include a meandering shaped region. For example, a user can insert the guidewire 100 into a large artery such as the femoral artery of a patient in a TAVR procedure.

At operation 904, the user may advance the guidewire through the blood vessel toward a target treatment tissue. This operation may involve navigating the guidewire 100 through the patient's vascular system, which may include the iliac artery, abdominal aorta, thoracic aorta, and aortic arch, or the like, ultimately crossing the target treatment tissue, such as the aortic valve in the case of a TAVR procedure.

At operation 906, benefited from the meandering shape of the guidewire 100, the user can engage the meandering shape against a wall of the blood vessel and the aortic valve so that the guidewire can be closer to the center of the blood vessel and aortic valve, potentially reducing the risk of the wire getting caught on calcified areas or in valve commissures.

At operation 908, the user may advance or withdraw the guidewire to adjust a position of the meandering shape relative to the target treatment tissue. This step allows for fine-tuning of the guidewire's position, which can change the angle of approach for the subsequent delivery system.

At operation 910, the user may advance the tissue delivery system at the changed angle of approach over the guidewire and across the target treatment tissue. This step involves using the guidewire as a rail to guide the delivery system (e.g., a TAVR delivery catheter) to the target site, benefiting from the optimized approach angle provided by the guidewire's positioning.

At operation 912, the user may advance, withdraw, or rotate the guidewire to align a distal ring marker of the tissue delivery system with an annular plane of the target treatment tissue. This final adjustment ensures proper alignment of the delivery system with the target tissue (e.g., aligning a TAVR device with the aortic annulus), which is crucial for successful deployment of the treatment device.

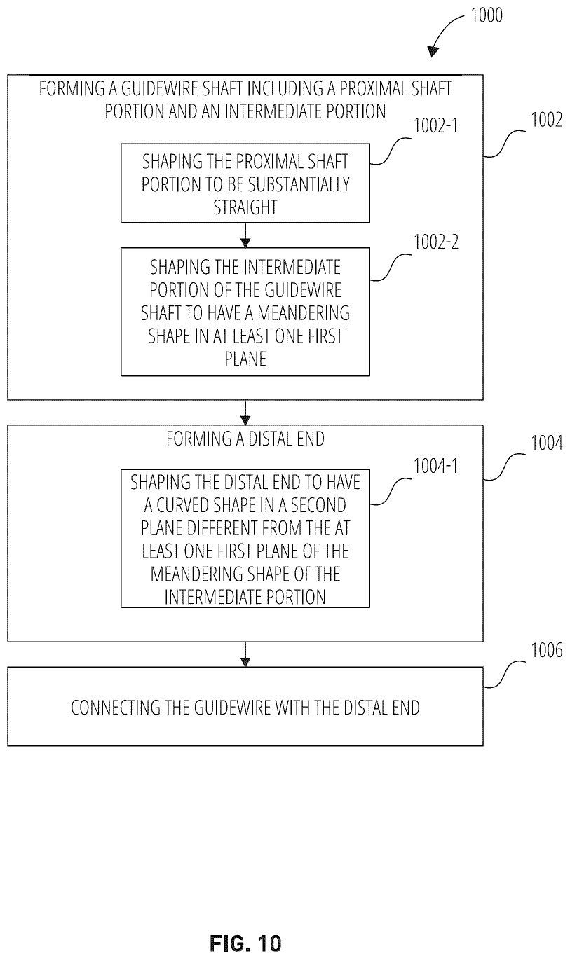

FIG. 10 illustrates a flowchart of a method 1000 of manufacturing a structural guidewire, in accordance with some examples.

At operation 1002, a guidewire shaft including a proximal shaft portion and an intermediate portion may be formed. The operation 1002 may include two sub-operations 1002-1 and 1002-2.

At operation 1002-1, the proximal shaft portion is shaped to be substantially straight. The proximal shaft portion can be constructed primarily of high-tensile stainless steel, such as 304V or 316LVM but this is not limiting. Any shape and material that can create the main body of the guidewire that provides the necessary stiffness and pushability can be chosen.

At operation 1002-2, the intermediate portion of the guidewire shaft is shaped to have a meandering shape in at least one first plane. The meandering shape can take various forms, such as a C-shape, S-shape, or a three-dimensional spiral shape. This unique shape may be helpful for centering the guidewire within blood vessels and facilitating navigation through complex vascular anatomy. The intermediate portion can be constructed primarily of high-tensile stainless steel, such as 304V or 316LVM. However, the intermediate portion can also be manufactured by other types of materials same or different from the proximal shaft portion. The intermediate portion can be manufactured independently with the proximal shaft portion and then combined or connected with the proximal shaft portion or they can be manufactured integrally.

At operation 1004, a distal end of the guidewire is formed. This operation 1004 may include a sub-operation 1004-1: shaping the distal end to have a curved shape in a second plane different from the at least one first plane of the meandering shape of the intermediate portion. This multi-planar configuration enhances the guidewire's ability to navigate three-dimensional vascular structures. The distal end can be made from nitinol, a nickel-titanium alloy, to allow for shape memory and flexibility. The curved shape can be a pigtail or S-shape.

At operation 1006, the guidewire shaft may be connected with the distal end. The connection can be achieved through several methods, for example:
1. A transition zone with tapered sections: the remote section of the intermediate portion and the proximal section of the distal end can be tapered and enclosed by an outer sleeve.
2. Welding techniques: methods such as friction welding or laser welding can be used to join the different sections.
3. Integral manufacturing: advanced techniques like laser cutting or 3D printing of metallic materials could create complex structures and transitions.

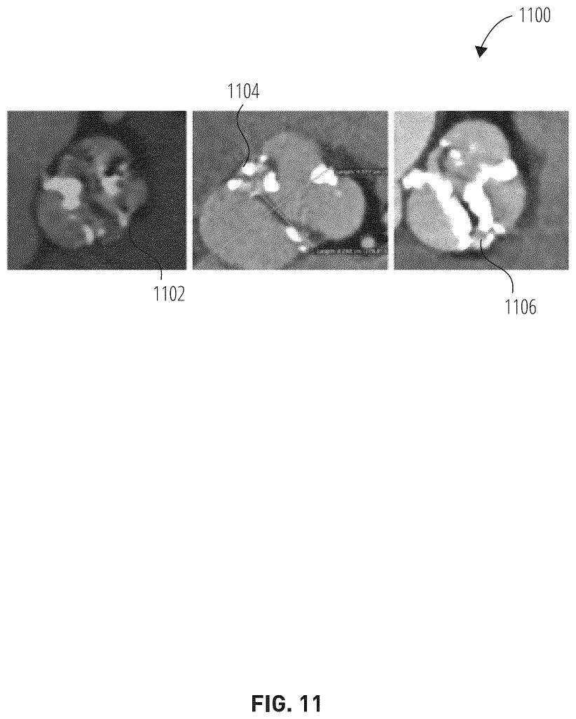

FIG. 11 illustrates various medical images 1100 indicating heavy calcification around aortic valves, in accordance with some examples. The images 1100 show cross-sectional views of aortic valves with varying degrees of calcification, highlighting the challenges faced during transcatheter aortic valve replacement (TAVR) procedures. The heavy calcification, particularly when extending to the bases 1102-1106 of adjacent leaflets, can cause wire bias towards the commissure base, potentially causing difficulty in the navigation and positioning of the guidewire and delivery system. The images 1100 underscores the need for the presently disclosed guidewire design with its unique meandering shape, which can help center the wire within the valve and move it out of the commissures, thereby facilitating easier crossing of heavily calcified valves during TAVR procedures.

Various examples of the disclosure are described herein. Reference is made to these examples in a non-limiting sense. They are provided to illustrate more broadly applicable aspects of the disclosure. Various changes may be made to the examples described without departing from the scope of the disclosure as defined by the appended claims. In addition, many modifications may be made to adapt a particular situation, material, composition of matter, process, process act(s) or step(s) to the objective(s), or scope of the present subject matter. Further, as will be appreciated by those with skill in the art that each of the individual variations described and illustrated herein has discrete components and features which may be readily separated from or combined with the features of any of the other several examples without departing from the scope of the present examples. All such modifications are intended to be within the scope of claims that may be associated with this disclosure.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only,” and the like in connection with the recitation of claim elements, or use of a “negative” limitation.

Without the use of such exclusive terminology, the term “comprising” or “including” in claims associated with this disclosure shall allow for the inclusion of any additional element, irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity. The breadth of the present disclosure is not to be limited to the examples provided and/or the subject specification, but rather only by the scope of claim language associated with this disclosure as defined by the appended claims.

1. A guidewire for intravascular procedures, comprising:
a guidewire shaft, comprising:
a proximal shaft portion being substantially straight; and
an intermediate portion integral with or connected to the proximal shaft portion, the intermediate portion having a meandering shape in at least one first plane; and
a distal end connected to the intermediate portion of the guidewire shaft, the distal end having a curved shape in a second plane different from the at least one first plane of the meandering shape of the intermediate portion.

2. The guidewire of claim 1, wherein the meandering shape is operable to centralize the guidewire in a valve having commissures and move at least a portion of the guidewire out of the commissures.

3. The guidewire of claim 1, wherein the curved shape of the distal end comprises a pigtail-shape or an S-shape.

4. The guidewire of claim 3, wherein the distal end comprises a core made of stainless steel or of nitinol.

5. The guidewire of claim 1, wherein the meandering shape of the intermediate portion comprises at least one of: a C-shape, or an S-shape.

6. The guidewire of claim 1, wherein the meandering shape of the intermediate portion comprises a three-dimensional spiral shape.

7. The guidewire of claim 6, wherein the three-dimensional spiral shape is offset from a longitudinal axis of the guidewire.

8. The guidewire of claim 1, wherein the intermediate portion has a length of 150-300 mm.

9. The guidewire of claim 1, wherein the guidewire shaft has a stiffness of 1.3-3.6 N/mm.

10. The guidewire of claim 1, wherein the guidewire has an overall length of 2500-3000 mm.

11. The guidewire of claim 1, wherein the guidewire has a diameter of 0.027-0.035 inches.

12. The guidewire of claim 1, wherein the second plane is perpendicular to one of the at least one first plane.

13. The guidewire of claim 1, further comprising a transition zone disposed between the intermediate portion and the distal end, the transition zone comprising:
a tapered remote section of the intermediate portion with a reduced diameter;
a tapered proximal section of the distal end with a reduced diameter; and
an outer sleeve enclosing and connecting the tapered remote section of the intermediate portion and the tapered proximal section of the distal end.

14. The guidewire of claim 13, wherein the tapered remote section of the intermediate portion and the tapered proximal section of the distal end each have a D-shaped cross-section, with flat surfaces of the D-shaped cross-sections facing each other and curved surfaces of the D-shaped cross-sections contacting the outer sleeve.

15. A method of advancing a guidewire through a blood vessel, the method comprising:
inserting a guidewire into a blood vessel of a human body, wherein the guidewire comprises a region of meandering shape;
advancing the guidewire through the blood vessel toward a target treatment tissue; and
facilitating centering of the guidewire in the blood vessel by engaging the meandering shape against a wall of the blood vessel or a wall of the target treatment tissue.

16. The method of claim 15, wherein the blood vessel is an aorta and the target treatment tissue is an aortic valve, the method further comprising:
advancing a transcatheter aortic valve replacement (TAVR) delivery system over the guidewire and deploying a prosthetic aortic valve into the aortic valve.

17. The method of claim 15, further comprising:
positioning a distal end of the guidewire in a heart chamber; and
positioning the meandering shape across the target treatment tissue,
wherein the target treatment tissue is an aortic valve, and the heart chamber is a left ventricle.

18. The method of claim 15, wherein the target treatment tissue is a valve, the method further comprising:
advancing or withdrawing the guidewire to adjust a position of the meandering shape relative to the valve to change an angle of approach for a valve delivery system, the angle of approach corresponding to the angle at which the valve delivery system approaches the valve during deployment; and
advancing the valve delivery system at a changed angle of approach over the guidewire and across the valve.

19. The method of claim 18, further comprising:
advancing, withdrawing, or rotating the guidewire to align a distal ring marker of the valve delivery system with an annular plane of the valve.

20. A method of manufacturing a guidewire for intravascular procedures, the method comprising:
forming a guidewire shaft comprising a proximal shaft portion and an intermediate portion, the forming of the guidewire shaft comprising:
shaping the proximal shaft portion to be substantially straight; and
shaping the intermediate portion of the guidewire shaft to have a meandering shape in at least one first plane; and
forming a distal end, comprising:
shaping the distal end to have a curved shape in a second plane different from the at least one first plane of the meandering shape of the intermediate portion.
